(19) United States
(12) Patent Application Publication
(10) Pub. No.: US 2013/0125883 A1
(43) Pub. Date: May 23, 2013

(54) LUNG VENTILATION DEVICE

(71) Applicants: Jorge BONASSA, Sao Paulo (BR); José Augusto CALVO LONARDONI, Osasco (BR); Orlando Luiz SANTARÉM DE ANDRADE, Sao Paulo (BR)

(72) Inventors: Jorge BONASSA, Sao Paulo (BR); José Augusto CALVO LONARDONI, Osasco (BR); Orlando Luiz SANTARÉM DE ANDRADE, Sao Paulo (BR)

(73) Assignee: INTERMED EQUIPAMENTO MEDICO HOSPITALAR LTDA., Cotia - Sao Paulo (BR)

(21) Appl. No.: 13/475,534

(22) Filed: May 18, 2012

(30) Foreign Application Priority Data
May 20, 2011 (BR) PI 1102142-0

(51) Int. Cl. A61M 16/00

(52) U.S. Cl. CPC A61M 16/0057; A61M 16/0051
USPC 128/202.22; 128/204.23

(57) ABSTRACT

The present invention refers to a lung ventilation device fitted with a patient monitoring and surveillance graphical interface comprising technical and functional characteristics that can reduce the cognitive load of the medical staff members in environments whose monitored beds require safe and efficient surveillance, principally for the identification and detection of parameters that are monitored at distance. More particularly, the present invention comprises a graphical interface whose configuration allows visualization at distance and interpretation of the main parameters associated with the patient, as well as includes means for facilitating the detection of the occurrence of critical alarms, even by an operator away from the patient.

### PRIORITY CLAIM AND CROSS-REFERENCE TO A RELATED APPLICATION

This application claims priority of Brazilian Patent Application Number PI 1102142-0 filed May 20, 2011, the entire contents of which are incorporated by reference herein.

### TECHNICAL FIELD

The present invention refers to a lung ventilation device fitted with a patient monitoring and surveillance graphical interface comprising technical and functional characteristics that can enhance patient surveillance and safety levels. More particularly, the graphical interface of the lung ventilation device comprises a configuration developed in a manner whereby the main parameters associated with the patient are presented in a way that allows easy visualization and interpretation at distance, lessening the cognitive load on the medical staff, and enhancing patient surveillance and safety. Furthermore, the present invention encompasses means for facilitating the detection of the occurrence of critical alarms, principally by an operator away from the patient.

### BACKGROUND OF THE INVENTION

Patients requiring artificial ventilation are generally connected to lung ventilators that cyclically deliver volumes of a mixture of air and oxygen through positive pressure. During mechanical ventilation, the patient presents changes in his clinical status, which must be continuously monitored and overseen by the healthcare staff.

In order to allow the proper adjustment of the equipment, as well as monitoring the patient, the ventilation equipment of the prior art presents a complex graphical interface, where the parameters that control the mechanical ventilation are monitored digitally and graphically, and are also associated with visual and audible alarms.

The main parameters that are monitored graphically are Pressure, Flow (inhalation and exhalation) and Volume (inhaled and exhaled) in the airway. The usual graphics are Pressure vs. Time, Flow vs. Time and Volume vs. Time. In addition are also relating Pressure vs. Volume and Flow vs. Volume.

Through morphology analysis of the plotted graphic is possible to assess the adequacy of the adjustment of the equipment and the interaction between the patient and the ventilator. It should be noted that the graphics do not usually allow fast and easy patient diagnosis. This is because, as may be noted by experts in the matter, interpretations of these graphics are possible only when diagnosed and analyzed by properly trained specialists not always available .

Beyond the graphics, the interfaces of the equipment of the prior art also present large number of information, including numerical data, messages, controls and alarms, whose complexity hampers their visualization, interpretation and analyses for taking decisions. According to the pioneering work by George A. Miller (The Magical Number Seven, Plus Minus Two Some Limits on Our Capacity for Processing Information. Psychological Review (1956) 2, 343-352), the number of items that a normal person can store in their working memory, which is required to process information, is around 7±2. More recently, according to Nelson Cowan (The magical number four in short term memory: A reconsideration of mental storage capacity. Behavioral in Brain Sciences (2000) 24, 87-185), the mental storage capacity is even smaller, at around 4±2.

Another point that integrates the Cognitive Load Theory (CLT), together with the work of Miller, is related to the manner in which information is presented. The use of different ways to present the same information, such as graphics and texts, requires high cognitive demand for association and interpretation of this information.

Examining the equipment known in the state of the art, it is apparent that these concerns, as well as functional and practical principles in this field of sciences have not been noted for the development of graphical interfaces fitted to patient control and monitoring equipment.

In addition to the inconvenient aspects mentioned above, another extremely relevant aspect is that the graphical interfaces of the equipment of prior the art are not properly designed for visualization at distance, requiring medical staff members to approach to the lung ventilation device, and it is only then that they can see the available information, including alarm status.

Thus, it is noted that the graphical interfaces of the equipment disclosed in the prior art are basically designed only to allow the operator to adjust the equipment and assess its interaction with the patient, by providing a large amount of information presented simultaneously on the screen. Furthermore, in case of a problem with the patient or the equipment, these interfaces were designed to trigger visual and audible alarms, although not adequately highlighting the visual indicator, due to the problems mentioned above. It is also important to stress that, as required by regulatory standards, audible signals offer only two variants—medium and high priority—distinguished by their harmonic composition. Thus, it is impossible for healthcare agents to distinguish between two alarms with the same priority but requiring different levels of urgency, such as disconnection of the respiratory circuit, which could lead to death or a rise in airway pressure because the patient coughed, with no major consequences.

It is important to stress that, particularly in intensive care environments, a medical staff is in charge of providing care for several patients, varying according to the hospital structure, with each patient being connected to various equipment, in addition to the lung ventilation device.

As is known for one skilled in the art, these intensive care environments consist of a series of beds, as well as a center called medical post, where the medical staff members are positioned to monitor the patients.

These beds are generally located more than three meters away from the central medical post. Therefore, the patients are monitored at a working distance that does not allow adequate visualization of the graphical interface, far less any identification of the main associated parameters and alarms, due to the configuration of the graphical interface and the disposition of the parameters presented.

It is important to stress that, in these environments, and due to the distribution of the above mentioned monitored beds by the medical staff, merely identification of which alarm, as well as the item of equipment and the patient, becomes an extremely arduous task, which is often difficult to complete immediately, imposing cognitive loads on the staff members that exceed acceptable limits.

This difficulty or limitation on detecting a dangerous condition considerably increases the risk of harm to patients.

Currently, the alarms are classified by the critical status of the condition, rated as low, medium and high priority alarms, with the medium and high critical status alarms being accompanied by a sound indicator, in addition to a visual indicator. The initial alert for the critical status of an alarm is initially sound-based. It is then necessary to identify which equipment/patient is in an alarm situation, and more specifically the type of alarm triggered. Occurs that some alarms classified under the regulatory standards as highly critical require faster responses than others, with the patient at risk of death.

Difficulties and resulting delays in meeting these situations greatly increase the risks involved. A recent specialized publication (Health Devices November 2010—ECRI Institute) noted that occurrences related to alarm responses are ranked among the top 10 hazards for patients. This paper highlights the saturation of the sensitivity of medical staff to sound alarms, resulting in deaths that could be avoided if the patients had been assisted on time.

In other words, due to the configuration of the interfaces, as well as constant alarms sounding simultaneously in the monitored environments, medical staff members do not have an efficient tool available that allows fast and immediate identification of endangered patients, as well as the reason why the alarm was triggered. Medical staff members must move over to the beds that might be reaching critical status, in order to identify the individual equipment whose alarm was triggered, as it is possible to determine what is happening only when close to the interfaces.

In conclusion, the interfaces have been designed to allow the operator to adjust the equipment, providing the largest possible amount of information on the screen, but they have clearly not been designed to allow and/or facilitate effective monitoring and surveillance of patients at the usual distance of the medical staff work.

Several patents describe ventilator interfaces including means to make them more user friendly, although it is noted that all of them provide means to assist ventilator setting but not effective patient vigilance.

Patent U.S. Pat. No. 6,269,812 and its continuation U.S. Pat. No. 7,036,504 describe a graphical interface and a display for ventilators of the prior art, comprising a touch screen permitting the adjustment and monitoring of the control parameters, the adjustment and monitoring of the alarms, and the graphic representation of the ventilation parameters. Said patents describe means to facilitate the adjustment of ventilator controls and alarms, although without offering means that help and/or facilitate the visualization and analysis of the ventilation parameters.

The application for Patent US 2009/0024008 proposes a method and a device for simplifying the diagnoses of patients on lung ventilation. It uses the dynamic representation of a stylized lung, whose dimensions are associated with the Volumes inhaled and exhaled by the patient during the respiratory cycle. However, due to the scale of the Figure, as well as the anatomical characteristics of each patient (sex, age, weight, pathology, height, etc.), it is not possible to obtain an adequate correlation and a resolution between the effective volume received by the patient and the alteration in size associated with the lung. This method also proposes an alteration in the lung profile and airway diameter of the image, according the respiratory mechanics of the patient. This visual alteration in the image profile reflects only one approximate qualitative aspect, since the verification of the progress of the patient mechanics requires the precise values of resistance and pulmonary compliance, generally obtained through a more detailed examination by a specialist. This absence of accurate information provided by the stylized lung image requires the association of other numerical and graphical data that, according to the prior filing in question, are associated on the same screen, increasing the cognitive load of the medical staff, together with difficulties in visualization and the complexity of the analyses for diagnosis.

As may be noted, the graphical interfaces of the equipment of the prior art disclose a complex configuration that is difficult for the medical staff to see, particularly when positioned at distance of the beds, meaning that it is not possible to engage in any immediate clinical analysis without walking over to the respective bed. Furthermore, due to the configuration of these graphical interfaces, which provide information through presenting a variety of parameters, data, graphics, and animated images, this underscores the need for high cognitive capacities among medical staff members, even when standing close to the equipment.

Furthermore, a simple correlation between a stylized animated image and the patient requires considerable cognitive effort, when attempting to obtain more accurate and reliable information on the clinical status of the patient. This is because the practitioners must pay attention to the image movements, while also ensuring an adequate interpretation of the animation in order to reach a diagnosis for the patient. To do so, a close view of the graphical interface is necessary.

Along these lines, it is noted that the lung ventilation equipment of the prior art present interfaces that are more appropriate for control and diagnosis, but not for the purposes of monitoring patient safety. The conventional safety system, with visual and audible alarms integrated to the control and monitoring interface, imposes high cognitive load on the healthcare staff, hampering the recognition and response to a deleterious situation for the patient.

Furthermore, the graphical interfaces of the equipment of the prior art do not allow the immediate detection of the critical aspects of monitored patients at usual wok distances.

### BRIEF DESCRIPTION OF THE INVENTION

Therefore, the purpose of the present invention is to provide a lung ventilation device that comprises a graphical interface configured in a manner that promotes effective patient monitoring and surveillance, being endowed with technical and functional characteristics that safely and efficiently resolve the problems and limitations of the equipment of the prior art.

More particularly, the purpose of the present invention is to provide a lung ventilation device that comprises a man-machine interface that can facilitate and promote effective patient monitoring and surveillance when caregiver is distant from the bed, improving the visualization and recognition of the main control parameters and the respective alarms, in order to enhance patient safety levels.

More particularly, the purpose of the present invention is to provide a lung ventilation device fitted with a patient monitoring and surveillance interface, where the main parameters associated with the patient are presented in a way that allows easy visualization at distance and interpretation of the clinical status of the patient, considerably reducing the cognitive load imposed on the medical staff members, thus considerably enhancing patient surveillance and safety.

Furthermore, the purpose of the present invention is to provide a lung ventilation device fitted with a graphical interface comprising the means for facilitating the detection of the occurrence of critical alarms, even by an operator away of the patient.

### BRIEF DESCRIPTION OF THE FIGURES

These and other purposes, technical effects and advantages of the lung ventilation device, fitted with a graphical interface for patient monitoring and surveillance, according to the present invention, will be apparent to the person skilled in the art, from the description made with reference to the attached schematic figures, which illustrate as exemplification and not limiting embodiments of the present invention, of which:

FIG. 1: illustrates a diagram of a mechanical lung ventilation system including the lung ventilation device, the respiratory circuit and a patient;

FIG. 2: illustrates a preferred representation of the graphical interface of the lung ventilation device, according to the present invention;

FIG. 3: illustrates a representation of an alternative configuration of the graphical interface of the lung ventilation device, according to the present invention;

FIG. 4: illustrates a representation of the alarm status indicator in a preferred configuration of the graphical interface of the lung ventilation device, according to the present invention.

FIG. 5: illustrates a representation of the critical alarm status indicator in a preferred configuration of the graphical interface of the lung ventilation device, according to the present invention.

### DETAILED DESCRIPTION OF THE INVENTION

In order to ensure an easier understanding of the configurable elements of the graphical interface of the lung ventilation device, according to the present invention, as well as its preferred embodiments, the numerical references will not be repeated in full in all the Figures, as this might hamper the understanding of some details illustrated therein.

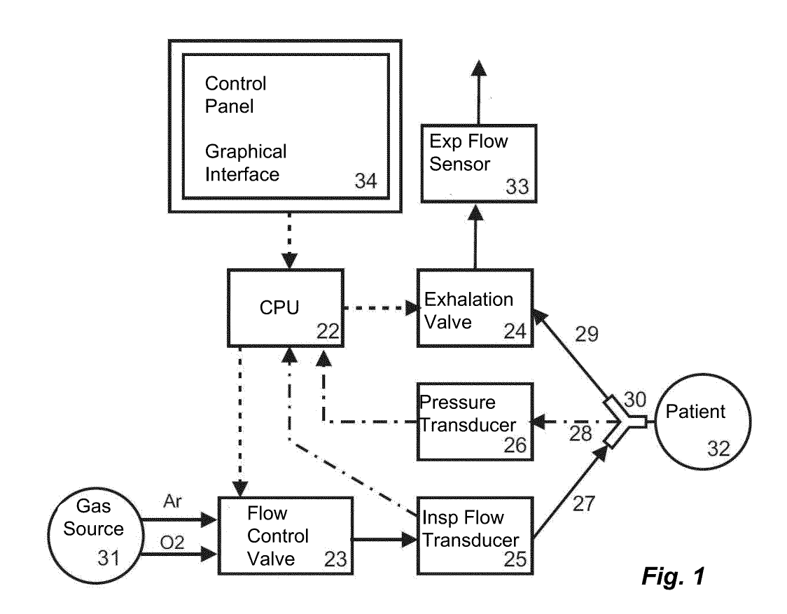

FIG. 1 schematically represents a typical ventilation system of the type contemplated by the present invention, comprising a flow and pressure control valve 23 connected to a gas source 31, which controls the inspiratory flow through an inflow tube 27 connected to the patient 32 through a “Y” shaped connector 30. The patient 32 exhales the gas through an outflow tube 29, connected to the other end of the “Y” shaped connector 30, as controlled by exhalation valve 24. The airway pressure of the patient is transmitted from the “Y” shaped connector 30 which is connected to a pressure transducer 26 through a tube 28.

The inspiratory flow is measured by the flow transducer 25 located downstream from the flow and pressure control valve 23. Both inspiratory flow and airway pressure signals coming from the flow transducer 25 and pressure transducer 26, together with the parameters adjusted through the control panel 34, are used by the central control unit 22 to servo control the flow and pressure valves 23 and the exhalation valve 24.

The flow and exhalation valves work in a bundle: when the flow valve is open, the exhalation valve is closed, promoting the patient to inhale; when the flow valve is closed, the exhalation valve is open, allowing the patient to exhale passively, respectively characterizing the inspiratory and expiratory phases of the respiratory cycle. The valve opening and closing times determine the Respiratory Rate, the Inspiratory Volume, and the Pressures during inspiratory and expiratory phases, according to the adjustments made on the control panel of the ventilation equipment. Monitoring of the Expiratory Volume is generally performed through an outflow sensor 33 connected to the expiration of the exhalation valve outlet 24. The Expiratory Volume measurement is considered more trustworthy, as it considers possible leaks in the respiratory circuit, thus reflecting the effective volume received by the patient.

In addition to contemplating the typical configuration of a ventilation system, as shown in detail in FIG. 1, the present invention is not limited to this configuration, encompassing alternatives that are commonly used in addition to future equivalent embodiments.

Mechanical ventilation itself refers to the process of supplying oxygenenriched gas actively through positive pressure, followed by passive removal through exhalation of the CO2-rich gas resulting from the breathing of the patient.

The total volume of gas inspired and expired by the patient during one minute, called the minute volume, summarizes the ventilator demand of the patient, and is associated with the efficacy of the lung ventilation.

The manner in which the lung ventilation device interacts with the patient is defined by different ventilation modes, allowing both the adequacy of the respiratory curve morphology as well as the type of respiratory cycles supplied to the patient. The curve morphology will be defined by the operator based on the pathology/protocol used. The types of respiratory cycle will be available as the patient recovers spontaneous ventilation capacity. There are three types of respiratory cycles:

i. Controlled cycle: respiratory cycle started, controlled and completed by the ventilation equipment;

ii. Assisted cycle: respiratory cycle started by the spontaneous effort of the patient, controlled and completed jointly by the patient and the ventilation equipment, assuring the controlled parameters;

iii. Spontaneous cycle: respiratory cycle started, controlled and completed basically by the effort of the patient, who may be partially assisted by the ventilation equipment.

During mechanical ventilation, the patient progresses from controlled cycle ventilation through to assisted cycles, finally reaching spontaneous cycles.

According to the purposes of the present invention, the control panel of the lung ventilation device includes a graphical interface, through which the following steps are taken:
Adjustment of the control parameters;
Monitoring of the graphic flow, pressure and volume indicators;
Digital monitoring of several parameters such as pressure, volumes, frequency, resistance, compliance, among others;
Adjustment and audible/visual signalization of the alarms;
Adjustment of service routines and configuration;
Monitoring trend data, alarm records, events, etc;
Control and monitoring of specific maneuvers during mechanical ventilation.

The various functions can be organized and/or grouped in specific screens, accessible directly or through menus.

Still according to the purposes of the present invention, the ventilation equipment comprises a graphical interface with processing that is independent of the processing required for the effective control of the mechanical ventilation, whereby a fault in the interface does not affect the functioning of the ventilator and vice versa. All the safety requirements are defined by applicable standards as well as risk analyses guiding the best construction options, forming part of the prior art.

Still according to the purposes of the present invention, the graphical interface may include a touch screen, in addition to a rotating or sliding control button, or some other technology performing equivalent adjustment functions that complies with the required purposes.

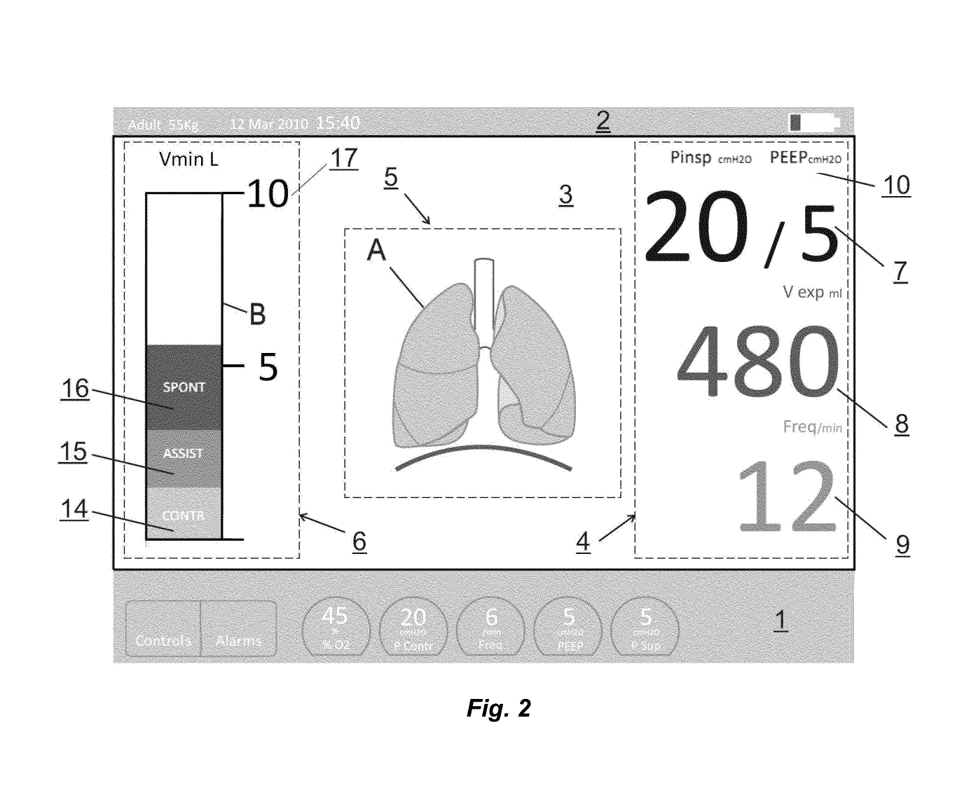

In a preferred configuration, the ventilation equipment comprises a graphical interface, which is fitted with a patient monitoring and surveillance screen, represented in FIG. 2 that displays details of the elements comprising the present invention.

As presented in FIG. 2, the patient monitoring and surveillance screen covers most of the graphic display area, preferably the central portion, with the adjacent areas available for access and control functions, as well as other indications such as date, time, battery level etc. Specifically for the preferred use of the present invention, on the lower part of the graphic display 1, the alarm and control access menus are arrayed in addition to fast access controls, covering a strip equivalent to 15% of the total area. The upper part of the graphic display 2, covers a strip whose area is equivalent to 5% of the total area, and is set aside for general information, such as date, hour, patient type etc., in addition to a central portion intended for the visual alarm indicator. The sizing of these adjacent areas is sufficient to contain the necessary elements, allowing its viewing at short distances.

The remaining area, which forms the central strip 3, comprises approximately 80% of the total area of the graphical display screen, and is set aside for the presentation of the patient monitoring and surveillance screen, containing elements to be viewed from further away, such as distance greater than 3 meters. Due to the dimensions of the graphical display used, its visualization may be increased proportionately.

More specifically, central strip 3 comprises a configuration divided into portions 4, 5, 6, intended respectively for the presentation of the numerical elements indicating the main ventilation parameters of the patient, an iconic element indicating the occurrence of the respiratory cycle of the patient, and a graphical element indicating the minute ventilation of the patient. These elements were selected as being sufficient to characterize the mechanical ventilation status, allowing the operator to watch the patient at distance with no need for interpretation, as he can be alerted by the simple view of each element.

More specifically, the three portions of 4, 5, and 6 of central strip 3 cover almost the same area, with approximately one third assigned to each portion.

In a preferred embodiment, the above mentioned portions are divided vertically, as illustrated in the appended figures, although, as must be appreciated by a person the skilled in the art, this proportionality and the array of portions 4, 5 and 6 may differ.

The configuration and proportionality of the above mentioned portions 4, 5, 6 were specifically designed to promote a reduction in the cognitive load of the medical staff members with regard to patient surveillance and monitoring, mainly in order to allow the immediate visualization of all the critical aspects of the patient by the staff members at the medical post that is located some distance away from the beds.

More particularly, for the purposes of the present invention, a 12″ display was considered, and as must be appreciated by a person the skilled in the art , displays of other sizes may be easily implemented.

Furthermore, for the purposes of the present invention, portion 4 presenting the numerical elements is preferably displayed on the right side of the patient monitoring screen, with the above mentioned portion 4 subdivided in a manner whereby the number of numerical elements is limited to four elements, namely: Inspiratory Pressure/Expiratory Pressure (PEEP—Positive End Expiratory Pressure) 7; Expiratory Volume 8 and Respiratory Rate 9. An Inspiratory and expiratory pressure 7 is presented side by side, due to the evident association between them, with the other elements arrayed vertically. Through these arrangements, adequately-sized fonts should be used as required for visualization at distance. For the present invention, the Calibri 100 font was used for the 12″ display, except for the expiratory pressure, which used the Calibri 80 font in order to highlight the Inspiratory pressure. The expiratory pressure is generally adjusted, and is not expected to alter for this reason, except in case of a fault in the equipment. On the other hand, depending on the type of ventilation, such as control volume ventilation, the Inspiratory pressure will rise or drop, depending on the clinical status of the patient.

Additionally, in order to further reduce the cognitive load imposed on the medical staff members, the three groups of parameters—pressure, volume and frequency—are presented in different colors, in order to ensure easy distinctions among the indicated parameters. In a preferred embodiment, for example, the pressures are presented in yellow (#FFFF00), the volume in pale blue (#00FFFF) and the frequency in white (#FFFFFF). In order to identify each parameter, titles 10 are shown under the respective parameter using fonts that can be read only from a short distance away. Specifically in the preferred embodiment of the present invention, the Calibri 17 font was used for the 12″ display. Thus, using the titles only for training and learning purposes, the parameters will be identified solely through their positions on the screen and the color of the indicated parameter.

Through this approach, the number of elements visible from some distance away can be reduced, maintaining the limit of only four elements and consequently reducing the cognitive load, assisted by the position and color codes.

Alongside the numerical elements in a central position on the screen, is disposed the portion 5, which presents a graphic/iconic element A that is intended to identify the respiratory cycle, specifically the start, duration and completion of an Inspiratory cycle, the exhalation phase and the cycle type (controlled, assisted or spontaneous).

In a preferred configuration, the graphic/iconic element represents the figure of the right and left lungs, the bronchial tree, the trachea and the diaphragm. The lung size is associated with the Volume inspired by the patient, meaning that the lung size increases during inspiration, through to the end of the inspiratory cycle, returning to the initial size at the end of exhalation. In order to relate the figure sizes on the patient monitoring and surveillance screen to the Volume inspired by the patient, the ideal body weight of the patient is used, as informed when starting up the ventilator. Based on the ideal body weight information, it is possible to estimate the maximum lung volume, which will be associated with the maximum size of the figure. The other volumes will be associated with the respective figure sizes. For example, it is common practice in medicine to establish a ponderal volume of 8 ml/kg in relation to the ideal body weight (normal weight of a person based on their height). Thus, a patient weighing 50 kg will have a recommended inhalation volume of 50×8=400 ml. The maximum Volume may be considered as a multiple of the recommended Volume, twice for example. Thus, the maximum size (100%) of the animated lung figure would correspond to n×Pi×Vp, where:
n is a multiple, in the present invention preferably between 2 and 3, although it may exceed these values;
Pi is the ideal weight, corresponding to the weight of a normal person based on their height;
Vp is the ponderal volume, the inspiratory volume for each kg of the ideal weight of the patient, in the present invention preferably between 6 and 10 ml/kg.

During exhalation, the figure size returns to the baseline volume dimension. This baseline dimension may be fixed or compensated automatically, as a function of the expiratory pressure used during ventilation. During mechanical ventilation, it is usual to maintain a minimum expiratory pressure (PEEP) higher than the atmospheric pressure, in order to avoid the collapse of the pulmonary alveoli. In proportion to the expiratory pressure level, the residual lung volume increases.

Thus, in a preferred configuration of the present invention, the baseline size of the lung figure is proportional to the residual lung volume at the measured expiratory pressure. The residual volume is calculated by multiplying the expiratory pressure by the lung compliance. Lung compliance is a datum monitored by the ventilator, which relates the variation between the inspiratory and expiratory pressures to the exhaled volume. The exact calculation falls within the domain of the prior art, and extends beyond the scope of the present invention. Thus, the baseline size of the Figure A of the lung is related to the calculated residual volume. The condition of expiratorypressure set at zero gives the minimum possible size condition for the figure A of the lung. This minimum size will be the same for all patients, as it is intended to increase the scale of the figure as much as possible for easier movement visualization during mechanical ventilation.

It is important to stress that perception of the size of this Figure A is not intended to determine accurately the Volume inspired by the patient, whose exact volume is indicated by numerical element 8 in portion 4. As mentioned previously, the main purpose of Figure/icon A of the animated lung is to identify the occurrence and duration of the respiratory cycle, as well as the cycle type.

According to this purpose, and specifically in the present invention, in addition to undergoing an alteration in size proportional to the Volume inspired/expired by the patient, the lung undergoes a color, in order to distinguish between an inhalation and exhalation phase during the respiratory cycle.

In a preferred configuration during an exhalation phase of the respiratory cycle, the lung color is grey (#666666). When an inhalation cycle begins, the lung color changes, depending on the type of respiratory cycle, as follows:
pink (#FEAAFE)—controlled cycle;
yellow (#FFFF00)—assisted cycle and
green (#00FF00)—spontaneous cycle.

The color change is easily perceived from some distance away, even at high breathing rates, as is case with neonatal patients. Particularly in these cases, where respiratory cycles are brief and volumes are small, the color code is the most effective resource for identifying the occurrence of an event. This is because a fast and simple alteration in the size or shape of the figure is hard to perceive from some distance away, imposing an additional cognitive load on the medical staff members.

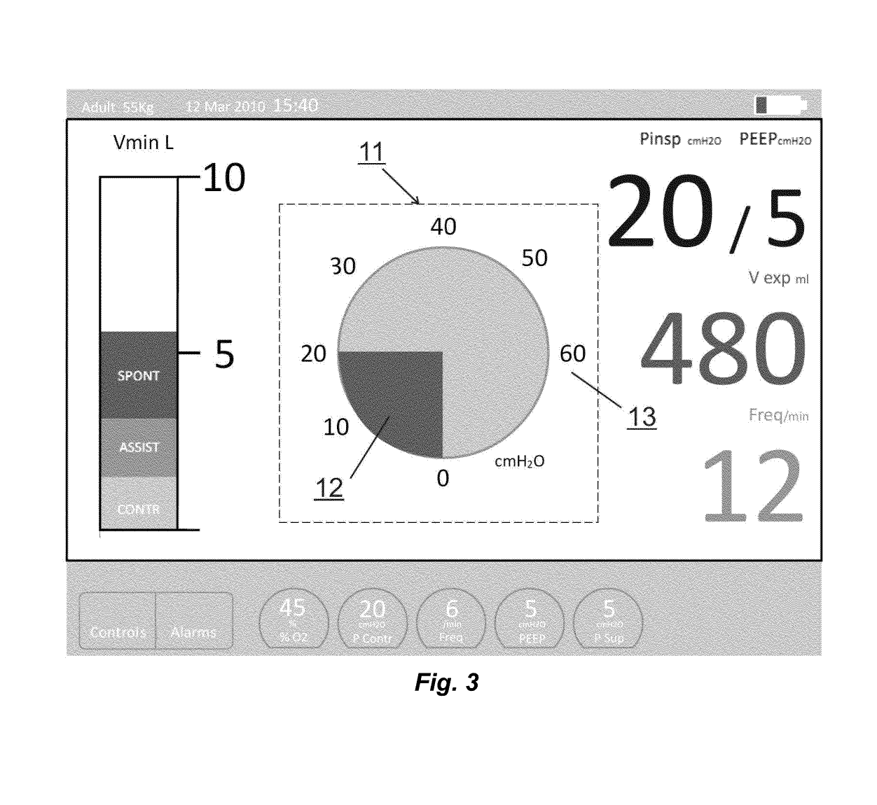

In an alternative configuration, as shown in FIG. 3, the figure of lung A may be substituted by a figure of a stylized analog manometer 11. The analog manometer measures the airway pressures through a pointer moving along a graduated scale. Pursuant to the purposes of the present invention, in order to highlight the pressurization of the airway during an inhalation phase of the respiratory cycle, the area of the circular sector defined by the position of the pointer and the start of the scale is filled with as color different from that of the manometer background, suppressing pointer 12. In this case, instead of an increase in the size of the lung as described above, the area of the circular sector, between the airway pressure and the atmospheric pressure, increases. Similar to the Figure of the lung, a color code is used to distinguish the exhalation phase from an inhalation phase. Similarly, in a preferred configuration of this embodiment, during the exhalation phase of the respiratory cycle, the color of the circular sector area is grey (#666666). When an inhalation phase begins, the area color changes, depending on the type of respiratory cycle, as follows:
pink (#FEAAFE)—controlled cycle;
yellow (#FFFF00)—assisted cycle and
green (#00FF00)—spontaneous cycle.

For the embodiment illustrated in FIG. 2, the primary parameter associated with the lung image is the volume, while in the alternative embodiment illustrated in FIG. 3, the parameter associated with the image of the manometer is the airway pressure. Depending on the type of ventilation used or the pathology of the patient, one alternative may be more advantageous than the other. For example, when using the “controlled volume ventilation” mode, it is expected that all cycles should have the same volume, whereby the lung image would not demonstrate any alteration in size between cycles. On the other hand, the use of the manometer image would reflect this variation through an alteration in the area due to the change in the respiratory mechanics of the patient, perceptible even from some distance away. These specific characteristics may be explored, in addition to the clear and unmistakable indication of the numerical elements.

Furthermore, the Value Scale 13 of the analog nanometer may be adjusted at various values, covering the physiological range, through touching the image on the touch screen, or through the scale adjustment menu, or by other equivalent means.

Finally, on the far left of the remaining area 3 of this screen, as illustrated in FIG. 2, portion 6 presents graphic element B indicating the minute ventilation of the patient. Preferably, the graphic element is a column type, indicating clearly and cumulatively the minute volumes resulting from each type of respiratory cycle. The blocked areas 14, 15 and 16, corresponding to each minute volume, are identified through a color code similar to that used for the animated lung image described above, as follows:
pink (#FEAAFE)—controlled minute volume 14;
yellow (#FFFF00)—assisted minute volume 15 and
green (#00FF00)—spontaneous minute volume 16.

In order to enhance the cognitive association, the positions of each type of minute volume hold defined places: spontaneous minute volume at the top, assisted in the middle, and controlled at the bottom. Furthermore, the Value Scale 17 may be adjusted among various values, covering the physiological range through touching the image in the touch screen or through a scale adjustment menu, or through other equivalent means. In a preferred configuration, the available scale values for the minute volume graphic are associated with the ideal weight of the patient, as mentioned previously, or in a more simplified manner they are related to the patient category: neonatal, pediatric or adult.

Graphic element B indicates the minute volume and distinguishes the contribution of each type of respiratory cycle, thus highlighting the participation of the patient's efforts to obtain the minute volume, clearly summarizing the status of the patient and the ventilation at any given time. The minute volume is the parameter that most directly reflects the mechanical ventilation, as it shows the quantity of gas moved and thus used for the gas exchanges. The minute volume is associated with the metabolic demand of the patient, and is thus a key patient monitoring parameter.

Additionally, another aspect of the present invention is related to the indication of the main alarms, which may be clearly identified from some distance away. The main alarms of the lung ventilation equipment are the following:
High/Low Inspiratory Pressure
High/Low Expiratory Pressure
High/Low Expiratory Volume
High/Low Respiratory Rate
High/Low minute volume

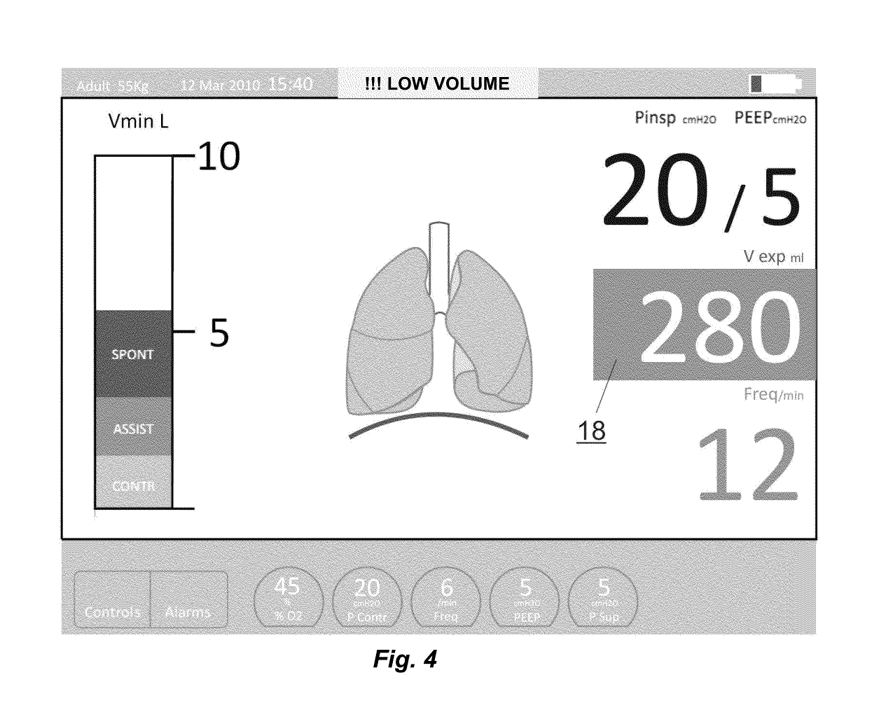

As may be noted, all these parameters are presented on the patient monitoring and surveillance screen of the ventilation equipment, as set forth in the present invention. As illustrated in FIG. 4, in the event of an alarm related to the numerical elements arrayed in portion 4 of the patient monitoring and surveillance screen, the respective element is highlighted with a flashing background in the alarm priority color 18. In compliance with the standard requirements, the alarm priorities are rated as low, medium and high priority, with the latter two being accompanied by a sound alarm. The color associated with the medium priority alarm is yellow, with red indicating high priority. According to the purposes of the present invention, the usual visual indicators of the alarms remain active, generally in the upper part of the screen, being added to the on-viewing of the patient monitoring screen, depending on their characteristics, allowing the parameter to be seen and identified from some distance away.

In addition to these alarms, two others are extremely critical and may result in the death of the patient, namely:
Patient Disconnected Alarm
Ventilator Inoperative Alarm

Should the patient become disconnected, several alarms are triggered, such as the low expiratory volume alarm, the low inspiratory pressure alarm and possibly others, such as the pressure drop alarm for the gases inlet. As may be noted, triggering all these alarms, particularly in such a critical condition, increases the cognitive load, without highlighting the most critical event, which is the disconnection and interruption of the ventilation of the patient. Another alarm that is equally important and high-risk is the ventilator inoperative indicator, which may be caused by countless factors, such as the absence of air/oxygen, operating defect etc. Once again, this condition is often confused with the others leading to the most critical event.

Detecting which alarm is active, as well as the reason why it was triggered, imposes a relatively high cognitive load on the medical staff members, as the practitioner must go over to the bed, identify the equipment whose alarm is sounding and only then assess the parameters in order to determine the alarm situation. All these steps require time, which is often not available, particularly for the critical alarms.

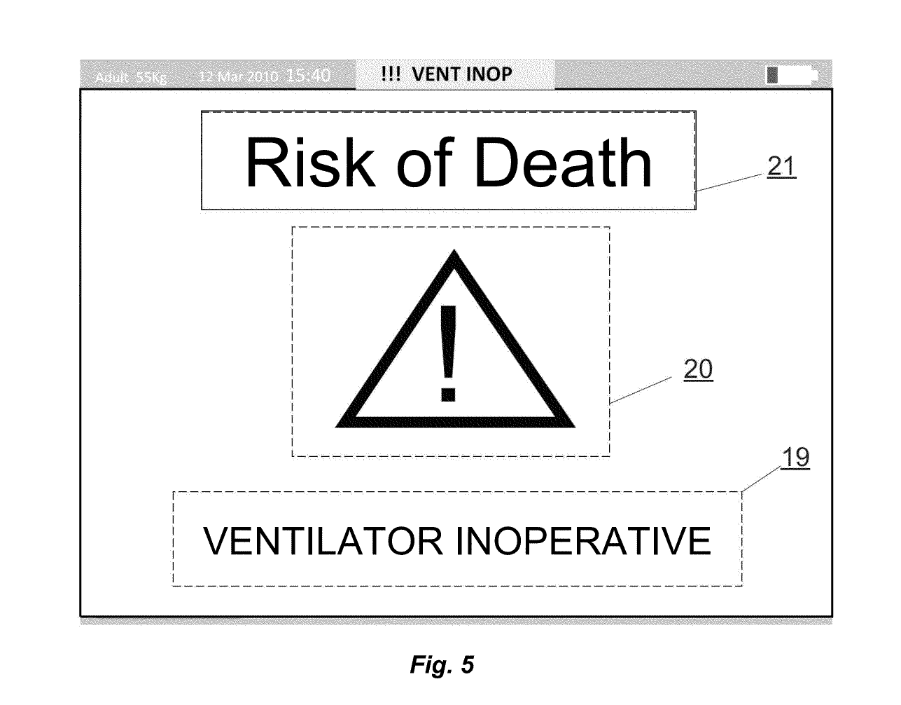

Thus, this condition is given particular consideration in the present invention, as shown in FIG. 5.

For the “Patient Disconnected” and “Ventilator Inoperative” alarms, the patient monitoring screen will present exclusively on a red background, the inscriptions of the corresponding alarms—“PATIENT DISCONNECTED” or “VENTILATOR INOPERATIVE” 19. Thus, there will be no competing information, immediately identifying which equipment/patient is at risk. These conditions may have other designations as appropriate to indicate the condition of imminent risk of death of the patient.

Alternatively, symbols or messages may be used or associated that highlight the critical status of the condition 20. The present invention also encompasses an alternative configuration where, in addition to identifying the alarm status, the associated risk of the patient is identified, also indicated in FIG. 5. For example, in case of disconnection, the associated risk, should the patient not breath spontaneously, is the “Risk of Death” 21. Other risks mapped according to a risk analysis may also be indicated, for example: Risk of Hypoxia, Risk of Brain Damage, Risk of Severe Damage, Risk of Moderate Damage, and Risk of Minor Damage. The identification of the risk associated with the alarm allows more effective awareness-heightening among the personnel involved in the services, including non-specialized staff, who may serve as monitoring support resources.

1-24. (canceled)

25. A lung ventilation device, comprising:
a control panel fitted with a patient monitoring and surveillance graphical interface,
a graphical display on the patient monitoring and surveillance graphical interface, the graphical display including a first portion with numerical elements indicating main ventilation parameters,
the graphical display including a second portion with an iconic element indicating the occurrence of a respiratory cycle, and
the graphical display including a third portion with a graphic element indicating a minute ventilation of a patient with the lung ventilation device.

26. The device of claim 25, wherein the first, second, and third portions are all located in a central strip of the graphical display.

27. The device of claim 26, wherein the central strip is about 80% of the total area of the graphical display.

28. The device of claim 26, wherein the number of numerical elements in the first portion is limited to four parameters.

29. The device of claim 28, wherein the parameters are Inspiratory Pressure, Expiratory Pressure, Expiratory Volume, and Respiratory Rate

30. The device of claim 29, wherein the inspiratory pressure and expiratory pressure numerical elements are arrayed side by side, and the expiratory volume and respiratory rate numerical elements are arrayed vertically below the inspiratory pressure and expiratory pressure numerical elements.

31. The device of claim 29, wherein the four numerical elements are shown in different respective colors.

32. The device of claim 25, wherein the iconic element further comprises signs indicating the type and status of the respiratory cycle.

33. The device of claim 31, wherein the signs indicate at least the start, duration and completion of an inhalation phase and the exhalation phase.

34. The device of claim 25, wherein the iconic element represents an image of the right and left lungs, the bronchial tree, the trachea and the diaphragm.

35. The device of claim 34, wherein the dimensions of the lung image represents the volumes inhaled and exhaled by the patient.

36. The device of claim 35 wherein the dimension of the lung image is determined based on the ideal weight of the patient, giving the maximum volume (Vmax) of the lung associated with the maximum size of the image of the iconic element.

37. The device of claim 36, wherein the maximum volume (Vmax) is a multiple (n) of the ponderal volume (Vp) based on the ideal weight (Pᵢ) of the patient, being represented by:
Vmax=n×Pᵢ×Vp, where:
n is a multiple, preferably between 2 and 3;
Pᵢ is the ideal weight, corresponding to the weight of a normal person for their height;
Vp is the ponderal volume, the inhalation volume for each kg of ideal weight of the patient, preferably between 6 to 10 ml/kg.

38. The device of claim 35, wherein baseline size of the lung image is proportional to the residual volume of lung for the measured expiratory pressure obtained by multiplying the expiratory pressure by the lung compliance calculated by the ventilator; being that at zero expiratory pressure, the lung image shows the minimum size, which will be the same for all patients.

39. The device of claim 35, wherein the iconic element is configured to display a predetermined color indicating the status between an inspiratory and expiratory phase of the respiratory cycle.

40. The device of claim 35, wherein the iconic element appears as a first predetermined color during the expiratory phase of the respiratory cycle, as a second predetermined color during the inspiratory phase when it is a controlled cycle, a third predetermined color during the inspiratory phase when it is an assisted cycle, and a fourth predetermined color during the inspiratory phases when it is a spontaneous cycle.

41. The device of claim 25 wherein the iconic element include an image of a stylized analog manometer.

42. The device of claim 25, wherein the third portion indicating minute ventilation further comprises a column type graphic element that presents in a distinct and cumulative way the minute volumes resulting from each type of controlled, assisted and spontaneous respiratory cycle, corresponding respectively to the controlled minute volume, the assisted minute volume and the spontaneous minute volume.

43. The device of claim 42, wherein the areas corresponding to each controlled, assisted and spontaneous minute volume appear in different predetermined colors.

44. The device of claim 25, wherein the graphic element comprises a scale, the device further comprising an input device, the input device configured to allow the scale of the graphic element to be adjusted.

45. The device of claim 44, where the scale is based on a patient category selected from neonatal, pediatric, or adult.

46. The device of claim 25, wherein the numerical elements of patient monitoring and surveillance graphical interface are configured to have flashing backgrounds that are color coded based on an alarm priority.

47. The device of claim 25, wherein the graphical interface is configured to display distinctive signs associated with the occurrence of risk alarms.
